(19) United States
(12) Patent Application Publication
(10) Pub. No.: US 2023/0398084 A1
(43) Pub. Date: Dec. 14, 2023

(54) Dermal Patch for Transdermal Administration of Ghrelin Pathway Blocker

(71) Applicant: Resarcion LLC, Wellesley, MA (US)

(72) Inventors: Pedram HAMRAH, Wellesley, MA (US); Reza MOLLAAGHABABA, Natick, MA (US)

(73) Assignee: Resarcion LLC, WELLESLEY, MA (US)

(21) Appl. No.: 18/239,557

(22) Filed: Aug. 29, 2023

Related U.S. Application Data
(63) Continuation of application No. 16/938,234, filed on Jul. 24, 2020.
(60) Provisional application No. 62/878,824, filed on Jul. 26, 2019.

(51) Int. Cl. A61K 9/70; C07K 16/26

(52) U.S. Cl. CPC A61K 9/7038; C07K 16/26

(57) ABSTRACT

Embodiments of the innovation relate to a dermal patch, comprising a substrate; a set of projections coupled to the substrate and configured to be at least partially insertable into skin, at least a portion of each projection of the set of projections comprising a biodegradable material; and a ghrelin blocker material encapsulated in the plurality of projections. The set of projections are coupled to the substrate via an adhesive that is configured to be dissolved within the skin after the patch is applied to the skin for a predetermined time, thus resulting in separation of the set of projections from the substrate. Once embedded in the skin, the protrusions can degrade and release the anti-ghrelin antibody encapsulated therein. The released anti-ghrelin antibody can find its way into the subject's circulatory system.

### RELATED APPLICATIONS

This patent application claims the benefit of U.S. Provisional Application No. 62/878,824, filed on Jul. 26, 2019, entitled, “A Dermal Patch for Transdermal Administration of Ghrelin Pathway Blocker,” the contents and teachings of which are hereby incorporated by reference in their entirety.

### FIELD

The present disclosure is generally directed to a dermal patch for providing controlled release of a ghrelin blocker into a subject's circulatory system for modulating the subject's appetite, and more particularly, to a dermal patch that provides controlled release of a ghrelin blocker into the subject's circulatory system.

### BACKGROUND

Ghrelin, also known as the “hunger hormone,” is a 28 amino acid peptide hormone that is produced by ghrelinergic cells in the gastrointestinal tract but also expressed in kidney, pituitary, pancreas, lymphocytes and brain. The active form of ghrelin is octanylated at Ser 3. It functions as a neuropeptide in the central nervous system to modulate appetite. Ghrelin stimulates gastric acid secretion and motility. Ghrelin levels significantly increase during fasting and decrease as a response to food intake. In addition to regulating appetite, ghrelin also plays a significant role in regulating energy homeostasis.

The ghrelin receptor (GHS-R1a) is a G-protein-coupled receptor that is most highly expressed in the hypothalamus. Outside the central nervous system, GHS-R1a can be found in the liver, in skeletal muscle and in the heart. It is well known that activating the GHS-R1a receptor with ghrelin induces an orexigenic state.

### SUMMARY

Conventional use of ghrelin blockers suffers from a variety of deficiencies. For example, there have been some efforts in using vaccination to modulate ghrelin level as a form of obesity prophylaxis and/or treatment. However, in humans, immunization against ghrelin has not shown promising results. For example, a randomized, double-blind and placebo-controlled trial with 87 obese patients aged 18-55 years with a body mass index between 30 and 35. The participants received four injections of 300 g of the vaccine or placebo at weeks 0, 4, 8, and 16. Despite a high production of ghrelin autoantibodies in the participants who received the vaccine, there was no additional weight loss achieved in comparison to the control group.

By contrast to conventional ghrelin blocker techniques, embodiments of the present innovation relate to a dermal patch for transdermal administration of a ghrelin pathway blocker.

In one embodiment, a dermal patch is disclosed, which comprises a support substrate having a plurality of shafts protruding above a surface thereof, said shafts being at least partially insertable into the skin when the patch is applied to the skin. A plurality of micro-needles are coupled to the shafts and are configured to be fully insertable into the skin, where the micro-needles include a biodegradable material. A ghrelin blocker, e.g., an anti-ghrelin antibody or antibody fragment, is encapsulated in said micro-needles. The micro-needles are attached to the shafts via an adhesive that is configured to be dissolved within the skin after the patch is applied to the skin for a predetermined time, thus resulting in separation of the micro-needles from the shaft. Once embedded in the skin, the micro-needles, which are formed of a biodegradable polymeric material, will degrade and release the ghrelin blocker encapsulated therein. The released ghrelin blocker can find its way into the subject's circulatory system.

The sizes of the micro-needles can be adjusted so as to modify the release profile of the ghrelin blocker embedded in the micro-needles into the subject's circulatory system. For example, an increase in the size of the micro-needles can result in a concomitant increase in the dissolution time of the micro-needles and hence the release time of the ghrelin blocker into the circulatory system.

As discussed in more detail below, the term “ghrelin blocker” as used herein refers to any of a small molecule, an antibody, an antibody fragment, an aptamer, an antibody catalyzer, or any other molecular species that can interact with the ghrelin hormone (e.g., acylated ghrelin) and/or one or more of its precursors, e.g., via binding to the ghrelin hormone, to modulate the biological function of ghrelin, e.g., to inhibit its normal interaction with the GHS-R1a receptor, e.g., to reduce its orexigenic effect. By way of example, a ghrelin blocker can be an antibody or antibody fragment that exhibits specific binding to ghrelin, acylated ghrelin or a precursor of ghrelin. The term “anti-ghrelin antibody fragment” as used herein refers to a fragment (e.g., Fab fragment) of an anti-ghrelin antibody. By way of example, the ghrelin precursor can be prepro-ghrelin.

The biodegradable material of the shafts is configured to degrade in the skin so as to release the ghrelin blocker materials into the subject's circulatory system.

In some embodiments, the biodegradable material can be poly-lactic acid (PLA), polyglycolic acid (PGA), poly-lactide-co-glycolide (PLGA), polydioxanone (PDS). Further, in some embodiments, the adhesive can include, for example, polyethylene glycol (PEG), polyethylene oxide (PEO), polyvinylpyrrolidone (PVP), gamma-polyglutamic acid (γ-PGA), gelatin and xanthan gum.

In some embodiments, the concentration of the ghrelin blocker materials in the biodegradable material can be, for example, in a range of about 0.1% to about 70% by weight, e.g., in a range of about 5% to about 60%, or in a range of about 10% to about 50%, or in a range of about 20% to about 40%, or in a range of about 25% to about 35%.

In some embodiments, the anti-ghrelin antibody can include anti-ghrelin immunoglobulin G. Anti-ghrelin antibodies suitable for use in the practice of the embodiments can be made using known techniques for generating polyclonal and monoclonal antibodies. For example, as is well known in the art, monoclonal antibodies can be generated using hybridoma-based technology. Human anti-ghrelin antibodies suitable for use in the practice of some of the embodiments are commercially available. For example, a human anti-ghrelin monoclonal antibody marketed by Abcam under the trade designation EPR20502 (ab209790) can be employed. As another example, ghrelin human monoclonal antibody (isotype IgG1λ) marketed by Enzo with UniProt ID Q9UBU3 can be used. In another example, anti-ghrelin antibody marketed by Novus Biologicals can be employed.

In some embodiments, an anti-prepro-ghrelin antibody marketed by Phoenix Pharmaceuticals, Inc. (e.g., Prepro (52-75)) can be employed to inhibit the production of ghrelin from the ghrelin precursor.

In other embodiments, a ghrelin blocker material can be a catalytic antibody that can catalyze hydrolysis of the serine ester of ghrelin to its inactive des-octanoyl form. By way of example, a catalytic antibody described in an article entitled “Catalytic antibody degradation of ghrelin increases whole-body metabolic rate and reduces refeeding in fasting mice,” published in Proc. Natl. Acad. Sci. USA 2008 Nov. 11; 105(45):17487-17492, which is herein incorporated by reference can be employed. This article discloses that monoclonal antibodies were obtained through immunization of mice with ghrelin phosphonate transition state analog, conjugated to the immunogenic carrier protein keyhole limpet hemocyanin (KLH), through a covalent link between the thiol moiety of the transition state analog and an N-maleimidomethyl cyclohexane-1-carboxylate cross-linker, resulting in hapten 2. The article explains that the design of this hapten was based on methodology that is well-established in the field of catalytic antibodies, in which phosphonate monoester 2 resembles the transition state of the hydrolysis reaction of the serine ester of ghrelin to its inactive des-octanoyl form. As a core antigen structure, the first 5 N-terminal amino acids were selected, partly based on studies that have evaluated truncated analogs of ghrelin, in which Ser³(octanoyl)-ghrelin (1-5) was shown to be the shortest structural analog to display activity similar to the native hormone.

The article further explains that another consideration was that the antibody combining site can host up to 8 amino acid residues; as such, the octanoate ester as found in natural ghrelin was truncated to a smaller 4-carbon chain appendage. In addition, the hapten with 2 isonipecotic acid (Isn) moieties were extended as a rigid linker to generate a more focused immune response, and a cysteine residue was included to enable a high-yield conjugation to KLH (see above).

Hapten 2 was synthesized on solid phase and was coupled to KLH through thioether conjugation chemistry; immunization of BALB/c mice with the immunoconjugate resulted in a panel of 19 monoclonal catalytic antibodies (mAbs) for analysis. All mAbs were purified from ascites, using ion-exchange and protein G affinity chromatography. A screening of the antibodies for catalytic hydrolysis of rat ghrelin to its des-octanoyl form with use of synthetic native rat ghrelin (experimental details are provided in SI Materials and Methods) indicated that several antibodies could accelerate the hydrolysis of native rat ghrelin. From this initial screen 3 mAbs demonstrated turn-over and were evaluated in greater detail.

As another example, a ghrelin blocker material can be the biostable aptamer 1-NOX-B11-2.

In some embodiments, the shafts can have a height that is sufficient to allow the shafts to penetrate the skin without injuring the subject. By way of example, in some embodiments, the height of the micro-needles can be in a range of about 400 microns to about 800 microns, e.g., in a range of about 500 microns to about 700 microns. In some embodiments, the shafts can be cylindrical with a diameter in a range of about 200 microns to about 400 microns. In other embodiments, the shafts can have a polygonal cross-sectional profile with a maximum cross-sectional dimension in a range of about 200 microns to about 400 microns.

In some embodiments, the micro-needles can have a conical shape with a height in a range of about 400 microns to about 800 microns and a base radius in a range of about 200 microns to about 400 microns. In some embodiments, the micro-needles can have a pyramidal shape with a height in a range of about 400 microns to about 800 microns and a maximum base dimension in a range of about 200 microns to about 400 microns.

In some embodiments, the above dermal patch can include an adhesive border surrounding the substrate that allows attaching the dermal patch to a subject's skin.

In a related aspect, a dermal patch is disclosed, which includes a substrate and a plurality of micro-needles that are coupled to the substrate, where the micro-needles encapsulate a ghrelin blocker material. The substrate can be a polymeric substrate. In some embodiments, the micro-needles can be attached to the substrate via an adhesive such that when in contact with the skin the micro-needles can be separated from the substrate and be embedded within the skin. The micro-needles are formed of a biodegradable material that is degraded once embedded in the skin to release the anti-ghrelin antibody encapsulated therein. The released anti-ghrelin antibody can find its way into the subject's circulatory system.

In another aspect, a dermal patch is disclosed, which includes a substrate having a plurality of micro-needles protruding above a surface thereof. The micro-needles and the rest of the substrate are formed as an integrated unit. In some embodiments, a liner can be attached, e.g., via an adhesive, to a surface of the substrate opposed to the surface from which the micro-needles protrude.

In a related aspect, a dermal patch is disclosed, which comprises a substrate having a plurality of shafts protruding above a surface thereof, the shafts being at least partially insertable into the skin, a plurality of micro-needles coupled to the shafts and configured to be fully insertable into the skin, the micro-needles comprising a biodegradable material, and a plurality of polymeric pockets distributed in the micro-needles, where the polymeric pockets encapsulate the ghrelin blocker materials. The micro-needles are attached to the shafts via an adhesive that is configured to be dissolved within the skin after the patch is applied to the skin for a predetermined time, thus resulting in separation of the micro-needles from the shafts.

In some embodiments, the micro-needles and the plurality of polymeric pockets are formed of different materials. In other embodiments, the micro-needles and the plurality of polymeric pockets are formed of the same material.

Further understanding of various aspects of the invention can be obtained by reference to the following detailed description in conjunction with the attached drawings, which are described briefly below.

### BRIEF DESCRIPTION OF THE DRAWINGS

The foregoing and other objects, features and advantages will be apparent from the following description of particular embodiments of the innovation, as illustrated in the accompanying drawings in which like reference characters refer to the same parts throughout the different views. The drawings are not necessarily to scale, emphasis instead being placed upon illustrating the principles of various embodiments of the innovation.

FIG. 1 schematically depicts a dermal patch according to an embodiment of the present disclosure.

FIG. 2 is top schematic view of the dermal patch depicted in FIG. 1.

FIG. 3 schematically depicts a dermal patch according to another embodiment of the present disclosure.

FIG. 4 schematically depicts a dermal patch according to yet another embodiment of the present disclosure.

FIG. 5 schematically depicts a dermal patch according to an embodiment of the present disclosure.

### DETAILED DESCRIPTION

The following detailed description refers to the accompanying drawings. The same or similar reference numbers may be used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

The present disclosure is generally related to dermal patches that can be employed to modulate a subject's appetite. As discussed in more detail below, in some embodiments, a dermal patch according to the present teachings can include a bio-degradable portion in which ghrelin blocker materials are encapsulated. Once in contact, or embedded in, a biological site such as the skin, the bio-degradable portion will naturally degrade to release the ghrelin blocker materials, at least a portion of which can find its way into the subject's circulatory system. In some embodiments, a ghrelin blocker material can bind to ghrelin and modulate its biological activity. For example, it can inhibit its interaction with the ghrelin receptor, thereby modulating the subject's appetite.

In some embodiments, an “antibody” refers to a polypeptide exhibiting specific binding affinity, e.g., an immunoglobulin chain or fragment thereof, comprising at least one antibody and antibody fragment. In some embodiments, an antibody comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody refers to an immunologically active, antigen-binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody.

In some embodiments, the term “antibody” also encompasses whole or antigen binding fragments of domain, or single domain, antibodies, which can also be referred to as “sdAb” or “VHH.” Domain antibodies comprise either V_{H} or V_{L} that can act as stand-alone, antibody fragments. Additionally, domain antibodies include heavy-chain-only antibodies (HCAbs). Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

In some embodiments, the term “anti-ghrelin antibody” refers to an antibody or an antibody fragment that can specifically bind to ghrelin so as to inhibit, or at least reduce, the binding affinity of ghrelin to a respective ghrelin receptor (Growth hormone secretagogue receptor (GHS-R). An anti-ghrelin antibody fragment refers to a Fab region of the anti-ghrelin antibody.

In some embodiments, the term “micro-needle” refers to a structure that extends from a proximal end to a sharp distal end, which is configured for contact with, and/or penetration into, the skin.

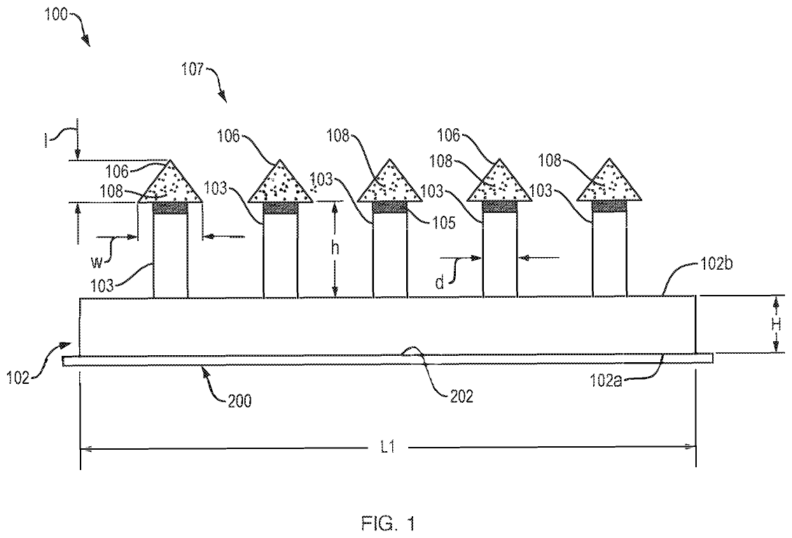

FIG. 1 schematically depicts a dermal patch 100 according to an embodiment of the present disclosure. Dermal patch 100 includes a substrate 102 extending from a bottom surface 102a to a top surface 102b configured for placement at a biological site such as the skin. The substrate 102 further includes a set of projections 107 that protrude from the substrate 102.

In one arrangement, the set of projections 107 include a plurality of shafts 103 that extend from the tope surface 102b of the substrate 102 and a plurality of biodegradable pyramidal-shaped micro-needles 106 that are attached to the top ends of the shafts 103 (for ease of illustration, the shafts 103 are not drawn to scale). Ghrelin blocker materials 108 are encapsulated within the micro-needles 106 for transdermal administration to an individual, as discussed in more detail below. In some embodiments, the ghrelin blocker materials 108 are embedded in pockets made of a polymeric matrix that is different from the polymeric matrix forming the micro-needles 106. In other embodiments, the ghrelin blocker materials 108 can be directly embedded within the polymeric matrix forming the micro-needles 106.

In some embodiments, the substrate 102 can have a rectangular prism shape with a length (L1) in a range of about 5 mm to about 20 cm, a height (H) in a range of about 1 mm to about 5 mm, and width (W) (perpendicular to the page and not shown in FIG. 1) in a range of about 5 mm to about 20 cm. It should be understood that these are merely examples of the shape and its dimensions. In one arrangement, the substrate 102 may be configured to have a number of other shapes, such as a prism with a non-rectangular base, e.g., circular, oval, etc., with other dimensions as suitable for a particular application.

Further, in some embodiments, the shafts 103 can have a cylindrical shape with a height (h) in a range of about 400 microns to about 900 nm, and a base diameter (d) in a range of about 200 microns to about 400 microns. In some other embodiments, the shafts 103 may be configured to haves a non-cylindrical shape, such as a prism shape with a cross section that is triangular, pentagonal, hexagonal, etc.

Further, the micro-needles 106 can have a length (l) in a range of about 250 microns to about 800 microns and a base width (w) in a range of about 200 microns to about 400 microns.

It should be understood that the substrate 102, the shaft 103, and the micro-needles 106 can have other shapes and dimensions so long as the patch 100 is capable of delivering the ghrelin blocker materials 108 to a subject's skin with minimal, if any, injury.

The substrate 102 can be formed of any suitable polymeric material, such as biodegradable polymeric materials. By way of example, in some embodiments, the substrate 102 can be formed of a biodegradable polyester polymer and copolymer, such as, polylactic acid (PLA), polyglycolic acid (PGA), poly-lactide-co-glycolide (PLGA) and polydioxanone (PDS) or derivatives thereof.

The micro-needles 106 can also be formed of a variety of biodegradable materials. By way of example, in some embodiments, the micro-needles 106 can be formed of biodegradable polymeric materials, such as chitosan, chitin, silk, carboxymethyl cellulose (CMC), chondroitin, collagen, and gelatin, among others.

In one embodiment, the micro-needles 106 can be attached to the shafts 100 via an adhesive 105 that can allow facile separation of the micro-needles 106 from the shafts 103 when the micro-needles 106 penetrate the skin. In this manner, the micro-needles 106 can be embedded in the skin Such an adhesive 105 can be coated on the top surfaces of the shafts 103. Placement of the micro-needles 106 onto the adhesive-coated shaft ends provides for connection of the micro-needles 106 to the shafts 103. Some examples of suitable adhesives 105 can include, without limitation, polyethylene glycol (PEID), polyethylene oxide (PEO), polyvinylpyrrolidone (PVP), polyvinyl alcohol (PVA), gamma-polyglutamic acid (γ-PGA), gelatin, maltose, xanthan gum, among others.

In some embodiments, the micro-needles 106 can be configured as pyramidal shapes having relatively sharp tips, which can facilitate the penetration of the micro-needles 106 into the skin. For example, during an application procedure, the micro-needles 106 can penetrate the stratum corneum and can be embedded in the epidermis, where they can naturally degrade to release the ghrelin blocker materials (e.g., anti-ghrelin antibodies) 108 encapsulated within the polymeric matrix of the micro-needles 106. In some embodiments, the penetration depth of the micro-needles 106 can be, for example, in a range of about 250 microns to about 800 microns.

It should be understood that the micro-needles 106 can be configured in a variety of shapes. For example, the micro-needles 106 can be cone-shaped, needle-shaped (e.g., in the shape of elongated, sharp cylinders), or of any other shape that can facilitate penetration of the micro-needles 106 into the skin.

In some embodiments, the concentration of the ghrelin blocker materials 108 in the micro-needles 106 can be, for example, in a range of about 1% to about 80%, for example, in a range of about 10% to about 70%, or in a range of about 20% to about 60%, or in a range of about 30% to about 50% by weight.

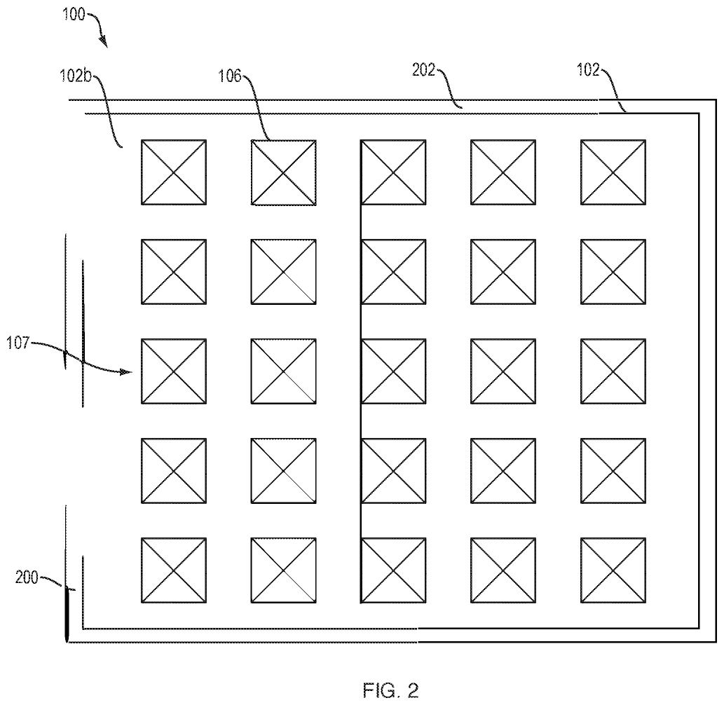

With additional reference to FIG. 2, the patch 100 can have a border 200 having a bottom surface to which an adhesive 202 is applied to facilitate affixation of the patch 100 to a skin site.

In use, the patch 100 can be applied to a skin site such that at least some of the shafts 103 and at least some of the micro-needles 106 at least partially, and in certain arrangements fully, penetrate through the skin. In this embodiment, the micro-needles 106 separate from the respective shafts 103 and become embedded in the skin, e.g., within the epidermis. Once embedded in the skin, the polymeric matrix can naturally decompose, and be absorbed and/or metabolized, thereby releasing the ghrelin blocker materials 108 encapsulated within the micro-needles 106.

Without being limited to any particular theory, at least a portion of the released ghrelin blocker materials 108 can find their way to the subject's circulatory system. Without being limited to any particular theory, at least some of the ghrelin blocker materials 108 can bind to circulating ghrelin. Such binding of the ghrelin blocker materials 108 to the circulating ghrelin can modulate biological activity of ghrelin within the subject. By way of example, the binding of the ghrelin blocker material 108 to ghrelin can inhibit ghrelin's activation of GHSR1a receptor. For example, the binding of the ghrelin blocker materials 108 to ghrelin can inhibit the binding of ghrelin with the GHSR1a receptor, or reduce the binding affinity of ghrelin with GHSR1a receptor, thereby modulating the activation of the GHSR1a receptor. This can in turn modulate, e.g., inhibit, the activation of the respective orexigenic neural circuits. In some embodiments, such modulation of the neural circuits can curb a subject's appetite.

According to some embodiments, in one method of manufacturing the dermal patch 100, in an initial step, a biodegradable polymeric solution is added to 1% of acetic acid solution to uniformly mix the mixture, and the mixture is then placed in deionized water for dialysis until achieving a pH value of about 6. In some embodiments, the mixture can be subsequently heated to evaporate additional water and achieve a desired concentration of the polymer, e.g., about 10 to 20 weight percent. This is followed by adding the anti-ghrelin antibodies 108 to the mixture and uniformly stirring the mixture. A portion of the mixture can then be placed on a mold. The mold covered with anti-ghrelin impregnated mixture can then be placed in a centrifuge machine and can be centrifuged at a rate, e.g., in a range of about 2000 to about 5000 rpm, at room temperature for a time duration, e.g., in a range of about 1 to about 2 hours. The mold has cavities in the shape of a pyramidal or conical micro-needles, which can receive the drug impregnated polymeric solution. The excess mixture on the exterior surfaces of the mold cavities can be removed. The mold can be subjected to further centrifugation to ensure that the mixture reaches the bottom of the cavities.

A pressing tool can then be used to further push the mixture containing the anti-ghrelin antibodies 108 into the cavities of the mold and additional steps of centrifugation and pressing can be performed.

Subsequently, a substrate 102 having shafts 103 coated with an adhesive 105 can be aligned and joined with the molded mixture, as disposed within the cavities of the mold. The combination of the substrate 102 and the mold can be baked, such as at a temperature of about 37° C., and the mold can be subsequently removed to provide the dermal patch 100.

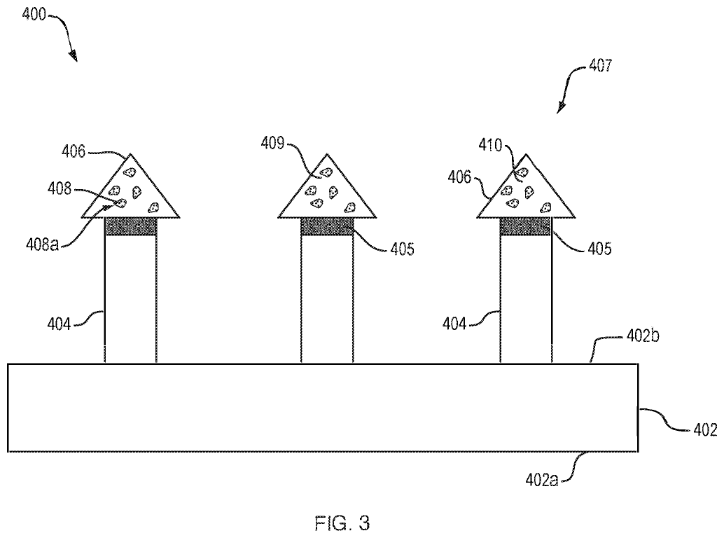

With reference to FIG. 3, in another embodiment, a dermal patch 400 according to the present disclosure can include a substrate 402 extending between a bottom surface 402a and a top surface 402b with a set of projections 407 extending from the top surface 402b. The set of projections 407 can include a plurality of shafts 404. Micro-needles 406 can be attached to corresponding shafts 404 by an adhesive layer 405.

A plurality of anti-ghrelin antibodies 408 can be distributed within a polymeric matrix or polymeric material 410 which form the micro-needles 406. For example, in this embodiment, at least a portion of the anti-ghrelin antibodies 408 are encapsulated by pockets 408a covered by a polymeric coating 409. The polymeric coating 409 material is different than the polymeric material 410 from which the micro-needles 406 are formed. In other words, in this embodiment, a plurality of polymeric coated pockets 408a are made from a polymeric material 409 that is distinct from the polymeric material 410 of the micro-needles 406, are loaded with the ghrelin blocker materials 408 and are distributed within the polymeric matrix 410 of the micro-needles 406.

In this embodiment, the polymeric coating 409 of the pockets 408a can degrade over a relatively longer time scale than the time scale associated with the degradation of the polymeric material 410 of the micro-needles 406. In some embodiments, the polymeric material 409 of the pockets 408a and/or molecules linked to the polymeric material 409 can be selected so as to allow masking of the pockets 408 from the subject's immune system.

For example, in this embodiment, a plurality of PEG molecules are coupled to the outer surface of the polymeric coating 409 of the pockets 408a. As discussed in more detail below, the PEG molecules can extend the circulation time of the pockets 408a loaded with the ghrelin blocker materials 408.

By way of example, in some implementations, the micro-needles 406 can be formed of polymeric materials, such as one or more of the polymers listed above, such as chitosan, chitin, silk, carboxymethyl cellulose (CMC), chondroitin, collagen, and gelatin, among others. Further, in some embodiments, the polymeric coating 409 of the pockets 408a can be formed of a polymer, such as polylactic acid (PLA), polyglycolic acid (PGA), poly-lactide-co-glycolide (PLGA) and polydioxanone (PDS) or derivatives thereof.

In use, the patch 400 can be applied to an individual's skin such that the micro-needles 406 at least partially, and in certain arrangements completely, penetrate through the skin. The micro-needles 406 separate from the respective shafts 404 and become embedded in the skin, e.g., in the epidermis. Once embedded in the skin, the polymeric matrix 406 can be naturally decomposed, absorbed or metabolized, thereby releasing the polymeric pockets 408a loaded with the ghrelin blocker materials 408, which find their way into the subject's circulatory system. The ghrelin blocker materials-loaded within polymeric pockets 408a can gradually degrade within the subject and can release their antibody (or other ghrelin blocker) cargo 408.

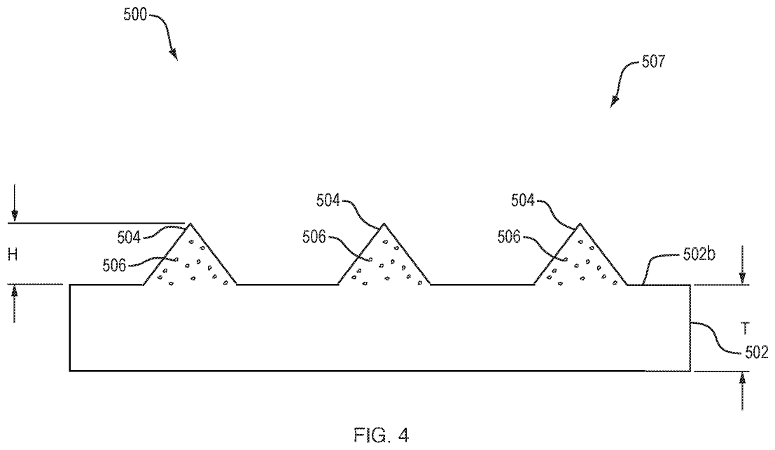

FIG. 4 schematically depicts a dermal patch 500 according to another embodiment, which includes a substrate 502 having projections 507 configured as a plurality of micro-needles 504 that protrude from a top surface 502b thereof. In this embodiment, the micro-needles 504 and the rest of the substrate 502 are formed as an integral unit, e.g., via molding. The micro-needles 504 can incorporate a plurality of ghrelin blocker materials 506 therein. The substrate 502 is formed of a biodegradable material, such as those discussed above, such that upon penetration of the micro-needles 504 into a subject's skin, the micro-needles 504 can degrade and release the encapsulated anti-ghrelin antibodies (or other ghrelin blocker materials) 506.

With continued reference to FIG. 4, in this embodiment, the substrate 502 is attached to an adhesive-coated liner (not shown) that facilitates attachment of the dermal patch 500 to a skin site. In some embodiments, a thickness (T) of the substrate can be, for example, in a range of about 1 mm to about 5 mm and a height (H) of the micro-needles 504 can be in a range of about 200 microns to about 800 microns, although other sizes may also be employed.

Without being limited to any particular theory, once released into a subject's circulatory system, the anti-ghrelin antibodies 506 can couple to ghrelin (e.g., acylated ghrelin) and/or a ghrelin precursor circulating in the subject's system to inhibit ghrelin and/or the ghrelin precursor from activating the ghrelin receptor.

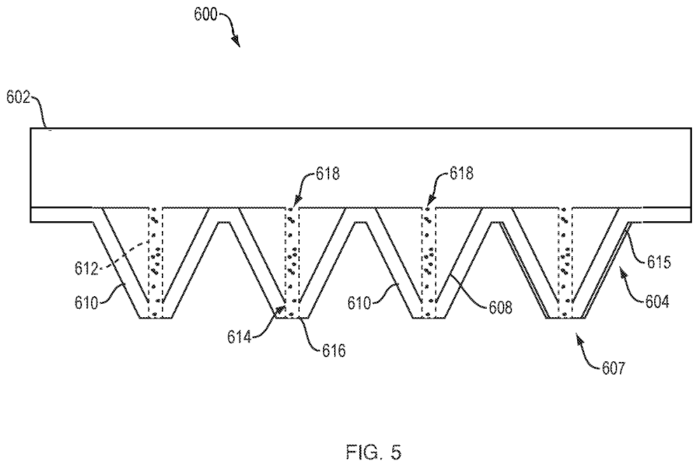

FIG. 5 schematically depicts another embodiment of a dermal patch 600 according to the present teachings, which includes a backing substrate 602 on which a polymeric layer 604 is disposed. The polymeric layer 604 includes a plurality of projections 607 (e.g., polymeric projections) that are sized and shaped to allow their insertion into the skin, e.g., in a manner discussed above in connection with the previous embodiments.

In this embodiment, the projections 607 include a polymeric body 608, which can be formed of one or more of the polymers disclosed herein, and a polymeric coating 610 that at least partially covers the polymeric body 608 of the projections 607. As discussed in more detail below, the polymeric coating 610 can protect the projections 607 as they are inserted into the skin and can be dissolved when the projections 607 are disposed within the skin.

In this embodiment, the projections 607 define a channel such as a hollow central channel 612 having an outlet 614 that is covered by a portion 616 of the polymeric coating 610. In this embodiment, each of the central channels 612 can be loaded with a composition containing at least one ghrelin blocker (such as an anti-ghrelin antibody) 618, such as those disclosed herein.

Upon insertion of the projections 607 into the skin, the polymeric coating 610 and covering portion 616 are dissolved within the skin, thereby unblocking the outlets 614 of the channels 612, which, in turn, results the release of the ghrelin blocker 618 from the channels 612 and into the tissue at the skin site. As discussed above, at least a portion of the released ghrelin blocker 618 can find its way to the individual's circulatory system to modulate ghrelin activity.

In this embodiment, the polymeric coating 610 is formed of PVP (polyvinylpyrrolidone), which is dissolved relatively quickly once inserted into the skin. In some embodiments, by adjusting a thickness of the polymeric coating 610, the rate of release of the ghrelin blocker 618 can be modified. By way of example, in some embodiments, the polymeric coating 610 can have a thickness in a range of about 0.5 mm to about 5 mm, e.g., in a range of about 1 mm to about 2 mm, though other thicknesses can also be used. Such controlled release of the ghrelin blocker material 618 can be useful titrating the blood concentration of the ghrelin blocker material 618.

In some implementations, the polymeric projections 607 and the backing substrate 602 are formed as a unitary structure, e.g., via injection molding or other suitable techniques. In some such embodiments, the polymeric projections 607 remain attached to the backing substrate 602 at least for a substantial portion of the use of the patch 600 on the skin. In other embodiments, the polymeric projections 607 can be formed separately from the substrate 602 and can be attached to the substrate 602, e.g., via an adhesive as discussed in connection with previous embodiments.

While in some embodiments, the polymeric projections 607 are formed of materials that can dissolve within the skin, in other embodiments, the polymeric projections 607 remain substantially intact once inserted into the skin. Further, in some embodiments, the polymeric projections 607 can be formed of different layers 615 of polymeric materials exhibiting different dissolution rates within the skin. By way of example, a polymeric projection 607 can include three layers, where the outer layer is formed of PVP (polyvinylpyrrolidone), a middle layer formed of polyethylene oxide (PEO), and an inner layer formed of polyvinyl alcohol (PVA).

In some embodiments, at least a portion (or all of) the ghrelin blocker 618 can be encapsulated in a plurality of polymeric particles (not shown), which are then disposed in the central channels 612 of the polymeric projections 607. Upon release into the skin, the polymeric particles can degrade and release the encapsulated ghrelin blocker 618, e.g., an anti-ghrelin antibody. While in some embodiments, the polymeric particles have substantially uniform sizes, in other embodiments, the sizes of the polymeric particles can vary such that some are degraded more quickly than others, thereby regulating the time release of the ghrelin blocker 618. The particles carrying the ghrelin blocker 618 can be formed of any suitable polymeric material, such as those discussed above for forming the microneedles. In some embodiments, the size of the polymeric particles (e.g., the diameter of the particles) can vary in a range of about 10 nm to about 10 mm, e.g., in a range of 100 nm to about 1 mm. The smaller particles can dissolve more quickly than the bigger particles and hence discharge their ghrelin blocker cargo 618 more rapidly. This can provide an extended time release of the ghrelin blocker 618 in the subject's circulatory system.

Further, in some embodiments, the particles carrying the ghrelin blocker cargo 618 can be formed of a variety of different polymeric materials that exhibit different dissolution rates within the skin and/or in the circulatory system. For example, in some such embodiments, some of the particles can be formed of PVP and some of the other particles can be formed of polyethylene oxide. Further, in some embodiment, both of the types of polymeric materials from which the particles are formed as well as the size of the particles can be varied to adjust the release time of the ghrelin into the subject's circulatory system.

In one example of a method for fabricating a dermal patch 100 according to the present teachings, micromolds can be fabricated using photolithography and known molding processes, such as those described in an article titled “Tapered conical polymer microneedles fabricated using an integrated lens technique for transdermal drug delivery” by Park JH et al. and published in Transactions on Biomedical Engineering 2007; 54(5); 903-913, which is herein incorporated by reference in its entirety. For example, a female micro-needle master mold can be formed in SU-8 resin by UV exposure to create micro-needles (e.g., pyramidal or conical). In some embodiments, the micro-needles 106 can exhibit a taper from their base to their tip. By way of example, the base and the tip can have a width of 300 microns and 25 microns, respectively, though other sizes can also be employed. In some embodiments, the lengths of the micro-needles 106 can be in a range of about 600 microns to about 800 microns, though any other suitable length can also be employed.

A male micro-needle master structure can be formed, e.g., of polydimethylsiloxane (PDMS). The male master structure can be coated with a gold layer (e.g., with a thickness of about 100 nm) to prevent adhesion of a second PDMS layer cured onto the make master structure to create a female PDMS replicate-mold.

In some embodiments, the micro-needle matrix can be formed by ultra-low viscosity carboxymethylcellulose (CMC), bovine serum albumin (BSV), and amylopectin in deionized water. Water can then be evaporated, e.g., by heating the mixture to a temperature in a range of about 60° C. to about 70° C., until a desired concentration of one or more solutes (e.g., CMC at about 27 wt %) is achieved, thereby forming a viscous hydrogel. One or more ghrelin blockers can be mixed with the hydrogel and subsequently, the hydrogel can be placed on a female micro-needle mold and can be subjected to centrifugation to fill the mold. The micro-needles 106 can then be released from the mold.

In use, such micro-needles 106 can be inserted into skin and can be dissolved within the skin to release their cargo of the ghrelin blocker 108.

Further details regarding materials and fabrication methods and materials that can be employed as informed by the present teachings to fabricate various embodiments of micro-needles according to the present teachings can be found, e.g., in an article titled “Dissolving Microneedles for Transdermal Drug Delivery,” by Jeon Woo Lee et al. published in Biomaterials, 2008 May: 29(13): 2113-2124, which is herein incorporated by reference in its entirety.

While several exemplary embodiments and features are described here, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. Instead, the proper scope of the embodiments is defined by the appended claims. Further, stating that a feature may exist indicates that the feature may exist in one or more embodiments.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or. Moreover, if these terms are used, a subset of a set may include one or more than one, including all, members of the set.

The foregoing description of the embodiments has been presented for purposes of illustration only. It is not exhaustive and does not limit the embodiments to the precise form disclosed. Those skilled in the art will appreciate from the foregoing description that modifications and variations are possible in light of the above teachings or may be acquired from practicing the embodiments. For example, the described steps need not be performed in the same sequence discussed or with the same degree of separation. Likewise various steps may be omitted, repeated, combined, or performed in parallel, as necessary, to achieve the same or similar objectives. Similarly, the systems described need not necessarily include all parts described in the embodiments, and may also include other parts not described in the embodiments. Accordingly, the embodiments are not limited to the above-described details, but instead are defined by the appended claims in light of their full scope of equivalents.

1.-19. (canceled)

20. A method of modulating a subject's appetite, comprising:
applying a dermal patch having a plurality of projections to a subject's skin such that the projections penetrate into the skin, wherein said dermal patch includes a ghrelin blocker incorporated in the plurality of projections at a concentration configured to modulate the subject's appetite,
wherein the projections include a biodegradable matrix material is degradable in the skin so as to release a sufficient amount of the ghrelin blocker into the skin such that the ghrelin blocker is introduced into the subject's circulatory system so as to modulate the subject's appetite.

21. The method of claim 20, wherein the ghrelin blocker comprises an anti-ghrelin antibody.

22. The method of claim 20, wherein the ghrelin blocker comprises an anti-ghrelin antibody fragment.

23. The method of claim 20, wherein the ghrelin blocker comprises an anti-ghrelin aptamer.

24. The method of claim 20, wherein the ghrelin blocker comprises an anti-ghrelin antibody catalyzer.

25. The method of claim 20, wherein the ghrelin blocker exhibits specific binding to ghrelin, acylated ghrelin or a ghrelin precursor.

26. The method of claim 25, wherein the ghrelin precursor is prepro-ghrelin.

27. The method of claim 20, wherein the concentration of the ghrelin blocker in the plurality of the projections is in a range of about 1% to about 80%.

28. The method of claim 20, wherein the concentration of the ghrelin blocker in the plurality of the projections is in a range of about 10% to about 70% by weight.

29. The method of claim 20, wherein the concentration of the ghrelin blocker in the plurality of the projections is in a range of about 20% to about 60% by weight.

30. The method of claim 20, wherein the concentration of the ghrelin blocker in the plurality of the projections is in a range of about 30% to about 50% by weight.

31. The method of claim 20, wherein the ghrelin blocker is configured to interact with any of circulating ghrelin and at least one of its precursors for modulating the subject's appetite.

32. The method of claim 20, wherein the ghrelin blocker comprises anti-ghrelin immunoglobulin G.

33. The method of claim 20, wherein the biodegradable matrix material comprises any of chitosan, chitin, silk, carboxymethyl cellulose (CMC), chondroitin, collagen, and gelatin.

34. The method of claim 20, wherein at least one of the projections of the dermal patch comprises a plurality of polymeric particles encapsulating the ghrelin blocker.

35. The method of claim 34, wherein said at least one of the projections comprises a channel in which the plurality of the polymeric particles is disposed.

36. The method of claim 34, wherein said plurality of polymeric particles exhibit different sizes.

37. The method of claim 34, wherein said plurality of polymeric particles are formed of different polymeric materials.

38. The method of claim 20, wherein said different polymeric materials exhibit different dissolution rates in the skin.
